(19) United States
(12) Patent Application Publication
(10) Pub. No.: US 2002/0156073 A1
(43) Pub. Date: Oct. 24, 2002

(54) Method for treating glaucoma IVB

(76) Inventors: Dilip Wagle, New York, NY (US); Martin Gall, Morristown, NJ (US); Stanley C. Bell, Narberth, PA (US); Edmond J. LaVoie, Princeton Junction, NJ (US)

Correspondence Address: ALLEN BLOOM, C/O DECHERT, PRINCETON PIKE CORPORATION CENTER, P.O. BOX 5218, PRINCETON, NJ, 08543-5218, US

(21) Appl. No.: 10/038,113

(22) Filed: Dec. 31, 2001

Related U.S. Application Data
(60) Provisional application No. 60/296,434, filed on Jun. 6, 2001.
(60) Provisional application No. 60/259,427, filed on Dec. 29, 2000.

(51) Int. Cl. A61K 31/541; A61K 31/5377; A61K 31/53; A61K 31/506; A61K 31/501; A61K 31/498; A61K 31/496; A61K 31/4439

(52) U.S. Cl. 514/227.8; 514/231.5; 514/252.01; 514/242; 514/255.05; 514/252.13; 514/317; 514/255.03; 514/336

(57) ABSTRACT

Provided, among other things, is a method of decreasing intraocular pressure or improving ocular accommodation in an animal, including a human, comprising administering an intraocular pressure decreasing or ocular accommodation improving amount of a compound of the formula I:

Het-Y (I)

This application claims the priority of U.S. Applications No. 60/296,434, filed Jun. 6, 2001, and 60/259,427, filed Dec. 29, 2000.

The present invention relates to methods for treating glaucoma or improving accommodation (i.e. the process by which the eye adjusts for vision at different distances), and to compounds and compositions for use in such treating. In one aspect, the present invention relates to a method of decreasing the intraocular pressure caused by glaucoma.

Diabetes is the major determinant to the development of visual disability and blindness in parts of the world unencumbered by causes related to malnutrition or infectious diseases. Retinopathy is the leading cause of blindness in diabetics and is a progressive, degenerative disease. Of the many risk factors believed to be associated with diabetic retinopathy, the level of glucose in the plasma has been widely investigated. It is well accepted that a lower incidence of retinopathy is associated with decreased plasma levels of glucose.

Ophthalmologic disorders in diabetes include opacification and glaucoma. As the occurrence of these indications is correlated with the persistent hyperglycemia of the disease. Although the incidence of glaucoma is significant in diabetic populations, glaucoma affects a substantial portion of the general aging population as well.

Primary open angle glaucoma occurs in approximately 4% of diabetics compared to 1.8% of the general population. The reasons for the increase in intraocular pressure that is observed in this disorder are not completely understood. The increase in intraocular pressure that characterizes glaucoma is likely caused by an impairment in the drainage of fluid from the eye at the trabecular meshwork since trabeculectomy restores, at least for a period of time, normal intraocular pressures. The origin of this impairment to fluid movement is currently unknown but may be related to a physical obstruction or restriction to movement of proteins that make up a sieving system in the trabecular meshwork. The trabecular meshwork functions as a sieving system that maintains a restricted flow of intraocular fluid from the eye. The result of excess restriction of this flow is a back pressure that causes increased intraocular pressure.

Replacement of the trabecular meshwork (trabeculectomy) remains an established surgical procedure for improving the filtering of intraocular fluid and for overall reduction of intraocular pressure. This remedy is invasive and of limited effectiveness, since pressure elevation frequently recurs after the procedures.

Current chronic pharmaceutical therapies impose a measure of risk on an already medically compromised patient population. The use of topical B-blockers may affect underlying cardiovascular disease, and carbonic anhydrase inhibitors (e.g. Diamox™) may cause metabolic acidosis. The use of pressure-lowering drugs will be affected by the state of renal disease in compromised elderly and diabetic patients. The drawbacks associated with current pharmaceutical therapies highlight an unmet medical need for a chronic pharmaceutical intervention that is distinct in mechanism of action from current therapies.

New strategies for pharmaceutical intervention in the treatment of glaucoma based upon new mechanisms of action need to be identified. In addition, pharmaceutical agents that decrease the intraocular pressure associated with glaucoma are needed. Also, the methods of improving accommodation provided by the invention allow one to avoid costly and burdensome optical solutions, such as the use of separate reading glasses or glasses with bifocal lenses.

### SUMMARY OF THE INVENTION

In one embodiment, the invention relates to a method of treating or ameliorating or preventing glaucoma, decreasing intraocular pressure or improving or ameliorating ocular accommodation in an animal, including a human, comprising administering an intraocular pressure decreasing or ocular accommodation improving amount of a compound of the formula I:

Het-Y (I)

wherein:

Het is a five or six membered heterocycle having a first ring nitrogen and optionally, a second or third ring nitrogen, with the remaining ring atoms being carbon, oxygen, or sulfur; provided that Het is not thiazole, imidazole, oxazole, or dihydro or tetrahydro analogs; and Y and other substituents on Het are defined below.

### DETAILED DESCRIPTION OF THE INVENTION

In accordance with the present invention a method is provided for the treatment of an animal, preferably a mammal, preferably a human with ophthalmologic disorders including glaucoma and reduced accommodation. Briefly the method of the present invention provides for a method of treatment of mammals with glaucoma or reduced accommodation that can be caused by age or certain age-related diseased states such as diabetes. The method provides for administration of classes of inhibitors of advanced glycation. The invention further provides for methods to monitor the improvement in the ocular condition during the course of the administration of compound.

The agents used in the invention are compounds formula I:

Het-Y (I)

wherein:

a. Het is a five or six membered heterocycle having a first ring nitrogen and optionally, a second or third ring nitrogen, with the remaining ring atoms being carbon, oxygen, or sulfur; provided that Het is not thiazole, imidazole, oxazole, or dihydro or tetrahydro analogs thereof,

b. Het can be substituted on carbon atoms with

1. one or more substituents independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl (which alkyl can be substituted with alkyloxyimino), cycloalkyl, dialkylamino, halo, hydroxy, (C₂-C₆)hydroxyalkyl, mercapto, nitro, sulfamoyl, sulfonic acid, alkylthio, alkylsulfonyl, alkylsulfinyl, alkylsulfonamido, trifluoromethyl, morpholin-4-yl, 4-[C₆ or C₁₀]arylpiperidin-1-yl, 4-[C₆ or C₁₀]arylpiperazin-1-yl, thiomorpholin-4-yl, piperidin-1-yl, Ar* {wherein, consistent with the rules of aromaticity, Ar* is C₆ or C₁₀ aryl or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring can be optionally fused to a substituted benzene, pyridine, pyrimidine, pyridazine, pyrazine, or (1,2,3)triazine (wherein the ring fusion is at a carbon-carbon double bond of Het)}, Ar*-alkyl, Ar*-O, Ar*SO₂—, Ar*SO—, Ar*S—, Ar*SO₂NH—, AR*NH, (N—Ar*)(N-alkyl)N—, Ar*C(O)—, Ar*C(O)NH—, Ar*NH—C(O)—, and (N—Ar*)(N-alkyl)N—C(O)— (in one embodiment, R^{a} and R^{b} are not acyloxyalkyl, alkenyl, (C₁-C₃)alkylenedioxy or allyl); or

2. two adjacent substitutions together with their ring carbons form a fused C₆ or C₁₀ aryl ring which aryl ring can be substituted as set forth below; or

3. two adjacent substitutions together with their ring carbons form a C₅-C₇ fused cycloalkyl ring having up to two double bonds including any fused double bond of the Het group, which cycloalkyl ring can be substituted by one or more of the group consisting of alkyl, alkoxycarbonyl, amino, aminocarbonyl, carboxy, fluoro, or oxo (in one embodiment, the fused cycloalkyl has no double bonds except any fused double bond of the Het group); or

4. two adjacent substitutions together with their ring carbons form a fused 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N; or

5. two adjacent substitutions together with their ring carbons form a fused five to eight membered fused heterocycle, wherein the ring fusion is at a carbon-carbon bond of Het, wherein the fused heterocycle consists of ring atoms selected from the group consisting of carbon, nitrogen, oxygen, sulfur, or S(O)ₙ, wherein S(O)n is 1 or 2 (in one embodiment, options 4. and 5. are omitted); and

c. Het can be substituted on ring nitrogen atoms with

1. hydrogen, alkyl, alkoxycarbonylalkyl-, Ar*, Ar*alkyl-, Ar*C(O)alkyl-, ArS*(O)alkyl-, Ar*S(O)₂alkyl-, so long as the ring nitrogen atoms are not quaternized;

2. amino; or

3. at most one nitrogen with oxido (—O⁻) to form an N-oxide; and

d. Y is substituted on a ring carbon adjacent to the first or second ring nitrogens and is

1. hydrogen, oxo, alkyl, mercapto, alkylthio, amino, amino(C₁-C₅)alkyl, or aminophenyl, wherein the amino of the latter three groups can be (a) substituted with

(a) Ar*,

(b) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂ or

(c) formyl or alkanoyl,

2. —NHC(O)(CH₂)ₙ—D—R^{e}R^{f}, wherein D is oxygen, sulfur or nitrogen, wherein when D is nitrogen n is 0, 1 or 2, but when D is oxygen or sulfur n=1 or 2, and R^{f} is present only when D is nitrogen,

wherein

(a) R^{e} is

(1) Ar*, or

(2) a group of the formula

Het°-

wherein Het^{δ} is independently the same as Het, or

(3) a C₃-C₈ cycloalkyl ring having up to one double bond with the proviso that the carbon linking the cycloalkyl ring to D is saturated, which cycloalkyl ring can be substituted by one or more alkyl-, alkoxycarbonyl-, amino-, aminocarbonyl-, carboxy-, fluoro-, or oxo-substituents tin one embodiment, multiple substituents are located on different carbon atoms of the cycloalkyl ring except in the case of alkyl, alkoxycarbonyl, and fluoro substituents, which can be located on the same or different carbon atoms), or

(4) hydrogen, (C₂-C₆)hydroxyalkyl, alkanoylalkyl, alkyl, alkoxycarbonylalkyl, alkenyl, carboxyalkyl (which alkyl can be substituted with alkoxyimino), alkoxycarbonyl, Ar*, or Ar*-alkyl-; and

(b) R^{f} is independently hydrogen, hydroxy(C₂-C₆)alkyl, alkanoylalkyl, alkyl, alkoxycarbonylalkyl, alkenyl, carboxyalkyl (which alkyl can be substituted with alkyloxyimino), alkoxycarbonyl, independently a group Ar* or Ar*-alkyl;

wherein aryl or Ar* in addition to any substitutions specifically noted can be substituted with one or more general substituents selected from the group of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*C(O)NH—, carboxy, carboxyalkyl, cycloalkyl, dialkylamino, halo, trifluoromethyl, hydroxy, (C₂-C₆)hydroxyalkyl, mercapto, nitro, morpholin-4-yl, thiomorpholin-4-yl, piperidin-1-yl, Ar*O—, Ar*-, Ar*-alkyl-, sulfamoyl, sulfonic acid, 1-pyrrolidinyl, piperidin-1-yl, 4-[C₆ or C₁₀]arylpiperidin-1-yl, and 4-[C₆ or C₁₀]arylpiperazin-1-yl (in one embodiment, these general substituents are selected from alkyl, amino, dialkylamino, 1-pyrrolidinyl, 4-[C₆ or C₁₀]arylpiperazin-1-yl. 4-[C₆ or C₁₀]arylpiperidin-1-yl, azetidin-1-yl, morpholin-4-yl, thiomorpholin-4-yl and piperidin-1-yl); and

heterocycles except those of Het or Ar*, can be substituted with, in addition to substitutions specifically noted, one or more general substituents selected from acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*-, Ar*-alkyl, carboxy, dialkylamino, fluoro, fluoroalkyl, difluoroalkyl, hydroxy, mercapto, 4-[C₆ or C₁₀]arylpiperidin-1-yl, 4-[C₆ or C₁₀]arylpiperazin-1-yl, (C₁-C₃)alkylenedioxy, oxo, sulfamoyl, and trifluoromethyl;

or a pharmaceutically acceptable salt of said compounds,

with the proviso that where the compound of formula I is administered to decrease intraocular pressure at least one compound of formula I administered in effective amount is not a triazole, thiadiazole, tetrazole or pyridotriazole substituted on a ring carbon sulfonamide (the amide of which can be substituted) that has carbonic anhydrase inhibiting activity.

In one embodiment. Het-Y is

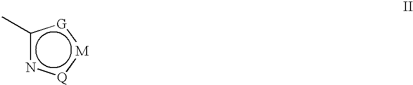

wherein G, M, and Q are selected from the group consisting of O, S, C—R^{h}, C—Rⁱ, and N—R^{g}, with the proviso that only one of G or Q can be O or S,

a. wherein R^{g}, is

(1) hydrogen, alkyl, alkoxycarbonylalkyl-, Ar*, Ar*-alkyl-, Ar*C(O)alkyl-, Ar*S(O)alkyl-, or Ar*S(O)₂alkyl-, so long as the ring nitrogen atoms are not quaternized; or

(2) amino or oxido (wherein N—R^{g} forms an N-oxide) and

b. wherein R^{h} or Rⁱ are

(1) independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl (which alkyl can be substituted with alkyloxyimino), cycloalkyl, dialkylamino, halo, hydroxy, (C₂-C₆)hydroxyalkyl, mercapto, nitro, sulfamoyl, sulfonic acid, alkylthio, alkylsulfonyl, alkylsulfinyl, alkylsulfonamido, trifluoromethyl, morpholin-4-yl, 4-[C₆ or C₁₀]arylpiperidin-1-yl, 4-[C₆ or C₁₀]arylpiperazin-1-yl, thiomorpholin-4-yl, piperidin-1-yl, Ar*, Ar*-alkyl, Ar*-O, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or;

(2) R^{h} and Rⁱ where adjacent, together with their ring carbons form a C₅-C₇ fused cycloalkyl ring having up to two double bonds including the fused double bond of the Het group, which cycloalkyl ring can be substituted by one or more of the group consisting of alkyl-, alkoxycarbonyl-, amino-, aminocarbonyl-, carboxy-, fluoro-, or oxo- substituents, except in the case of alkyl, alkoxycarbonyl, and fluoro substituents, which can be located on the same or different carbon atoms;

(3) R^{h} and Rⁱ where adjacent, together with their ring carbons form a fused C₆ or C₁₀ aryl ring;

(4) R^{h} and Rⁱ where adjacent, together with their ring carbons form a fused five to eight membered fused heterocycle, wherein the ring fusion is at a carbon-carbon bond of Het, wherein the fused heterocycle consists of ring atoms selected from the group consisting of carbon, nitrogen, oxygen, sulfur, or S(O)n, wherein S(O)ₙ is 1 or 2; or

(5) R^{h} and Rⁱ where adjacent, together with their ring carbons form a fused 5- or 6-membered heteroaryl ring containing at least one and up to three atoms of N for the 6-membered fused heteroaryl rings and from one to three atoms of

N or one atom of O or S and zero to two atoms of N for the 5-membered fused heteroaryl rings.

The compounds of formula II can be such that:

a. Q is N;

b. G is N—R^{g};

c. M is C—R^{h}; and

d. Y is amino, amino(C₁-C₅)alkyl, or aminophenyl, wherein the amino of all three groups can be substituted with

(1) Ar*,

(2) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂ or

(3) formyl or alkanoyl.

The compounds of formula II can also be according to the following:

| | |
|---|---|
| 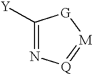 | 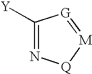 |
| wherein G is O, S, N—R^{g}; | wherein G is N or C—R^{h}; |
| M is N or C—R^{h}; or | M is N or C—R; and |
| Q is N or C—Rⁱ. | Q is O, S, or N—R^{g}. |
| 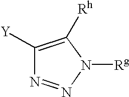 | |

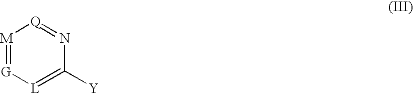

wherein Q, M, G and L are independently N⁻, C—R^{j}, C—R^{k}, C—R^{l}, or C—R^{m}, with the proviso that there are 1 to 3 N atoms in the ring, wherein R^{j}, R^{k}, R^{l} and R^{m} are

a. independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl (which alkyl can be substituted with alkyloxyimino), cycloalkyl, dialkylamino, halo, hydroxy, (C₂-C₆)hydroxyalkyl, mercapto, nitro, sulfamoyl, sulfonic acid, alkylthio, alkylsulfonyl, alkylsulfinyl, alkylsulfonamido, trifluoromethyl, morpholin-4-yl, 4-[C₆ or C₁₀]arylpiperidin-1-yl, 4-[C₆ or C₁₀]arylpiperazin-1-yl, thiomorpholin-4-yl, piperidin-1-yl, Ar*, Ar*-alkyl, Ar*-O, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—;or

b. where two of R^{j}, R^{k}, R^{l} or R^{m} are adjacent, together with their ring carbons form a C₅-C₇ fused cycloalkyl ring having up to two double bonds including the fused double bond of the Het group, which cycloalkyl ring can be substituted by one or more of the group consisting of alkyl-, alkoxycarbonyl-, amino-, aminocarbonyl-, carboxy-, fluoro-, or oxo- substituents, wherein multiple substituents are located on different carbon atoms of the cycloalkyl ring, except in the case of alkyl, alkoxycarbonyl, and fluoro substituents, which may be located on the same or different carbon atoms; or

c. where two of R^{j}, R^{k}, R^{l} and R^{m} are adjacent, together with their ring carbons form a fused C₆ or C₁₀ aryl; or

d. where two of R^{j}, R^{k}, R^{l} and R^{m} are adjacent, together with their ring carbons form a fused five to eight membered fused heterocycle, wherein the ring fusion is at a carbon-carbon bond of Het, wherein the fused heterocycle consists of ring atoms selected from the group consisting of carbon, nitrogen, oxygen, sulfur, or S(O)ₙ, wherein n is 1 or 2; or

e. where two of R^{j}, R^{k}, R^{l} and R^{m} are adjacent, together with their ring carbons form a fused 5- or 6-membered heteroaryl ring containing at least one and up to three atoms of N for the 6-membered fused heteroaryl rings and from one to three atoms of N or one atom of O or S and zero to two atoms of N for the 5-membered fused heteroaryl rings.

These compounds of formula III can be such that:

a. wherein L is N;

b. G is C—R^{j};

c. M is C—R^{k};

d. Q is C—R^{l}; and

e. Y is amino, amino(C₁-C₅)alkyl, or aminophenyl, wherein the amino of all three groups can be substituted with

(1) Ar*,

(2) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂ or

(3) formyl or alkanoyl.

Or, these compounds of formula III can be such that:

a. wherein L and Q are N;

b. G is C—R^{j};

c. M is C—R^{k}; and

d. Y is amino, amino(C₁-C₅)alkyl, or aminophenyl, wherein the amino of all three groups can be substituted with

(1) Ar*,

(2) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂, or

(3) formyl or alkanoyl.

In addition to the methods, compounds, and compositions thereof described herein, the invention provides methods or use in the treatments of the invention, or in the manufacture of a medicament for such therapeutic use.

Primary open angle glaucoma is characterized by an increase in intraocular pressure. The condition of open angle glaucoma is characterized by an increase in the pressure within a person's eye or eyes, called the intraocular pressure. The normal pressure is about 15 mmHg. Elevated pressures of 20-30 mm Hg create a strong risk of damage to the optic nerve and blindness.

Glucose reacts with proteins by a non-enzymatic, post-translational modification process called non-enzymatic glycosylation. The resulting sugar-derived adduct, the advanced glycosylation end product (AGE), matures to a molecular species that is reactive, and can readily bond to amino groups on adjacent proteins, resulting in the formation of AGE cross-links between proteins.

It has now been found that certain compounds that inhibit the formation of such sugar-derived adducts, or in some cases are believed to deactivate such adducts or break resulting crosslinks, can reduce intraocular pressure or ameliorate a trend towards elevated pressure.

Structural matrix proteins isolated from tissues of diabetics and aged individuals are more highly crosslinked than those from nondiabetics or younger individuals and are more resistant to both enzymatic and chemical hydrolysis in vitro. It is this cross-linked state of proteins that is believed to cause stiffness of tissues. The cleavage of AGE cross-links between proteins can provide a mechanism-based therapy for restoration of normal tissue function. An agent that cleaves AGE cross-links between proteins or inhibits their formation can restore more normal sieving function and movement to the trabecular meshwork.

In accordance with the present invention, methods for administering pharmaceutical compositions containing compounds have been developed for the treating glaucoma, intraocular pressure associated with glaucoma, and reduced accommodation. These agents are nitrogen containing five and six-membered heterocycles of the formula I as shown in the Summary section above.

The heterocycles (Het) of the invention can be aromatic or non-aromatic. In those embodiments of the invention wherein Het contains a sulfur atom in the ring and is not aromatic, the sulfur atom can exist in various oxidation states as —S(O)ₙ—, where n is 0, 1, or2.

Preferred nitrogen containing five-membered ring heterocycles (Het) of the invention include pyrazole, isoxazole, isothiazole, (1,2,4)triazole, (1,3,4)thiadiazole. The invention does not include thiazoles, oxazoles, imidazoles, or dihydro or tetrahydro analogs thereof Preferred nitrogen containing six-membered ring heterocycles (Het) of the invention include pyridine, pyridazine, pyrazine, and pyrimidine. Preferred compounds of the invention also include benzo-fused analogs of the foregoing. Het is therefor preferably pyrazole, isoxazole, isothiazole, (1,2,4)triazole, (1,3,4)thiadiazole, pyridine, pyridazine, pyrimidine, indazole, 1,2-benzisoxazole, 2,1-benzisoxazole, 1,2-benzisothiazole, 2,1 -benzisothiazole, quinoline, isoquinoline, phthalazine, cinnoline, quinoxaline, or quinazoline.

The alkyl, and alkenyl groups referred to below include both C1 to C6 linear and branched alkyl and alkenyl groups, unless otherwise noted. Alkoxy groups include linear or branched C1 to C6 alkoxy groups, unless otherwise noted.

“Ar*” (consistent with the rules governing aromaticity) refers to a C₆ or C₁₀ aryl, or a 5 or 6 membered heteroaryl ring. The heteroaryl ring contains at least one and up to three atoms of N for the 6 membered heteroaryl ring. The 5 membered heteroaryl ring contains; (1) from one to three atoms of N, or (2) one atom of O or S and zero to two atoms of N. The aryl or heteroaryl is optionally substituted as set forth below. Nonlimiting examples of heteroaryl groups include: pyrrolyl, furanyl, thienyl, pyridyl, oxazolyl, pyrazolyl, pyrimidinyl, and pyridazinyl.

“Ar*” can be fused to either a benzene, pyridine, pyrimidine, pyridazine, or (1,2,3) triazine ring.

As used herein, C6 or C10 aryl groups are monocyclic or bicyclic.

In certain embodiments of the invention, Het can contain two adjacent substitutions on carbon atoms that together with their ring carbons (the carbons of Het joining the adjacent substituents) form a five to eight membered fused heterocycle (i.e. a bicyclic heterocycle is formed). In these embodiments the fused heterocycle is preferably not aromatic. Particular compounds within these embodiments contain sulfur atoms in the fused heterocyclic ring. These sulfur atoms in these particular compounds can exist in various oxidation states, as S(O)ₙ, where n is 0,1, or 2.

In certain embodiments of the invention, Het can contain two adjacent substitutions on carbon atoms that together with their ring carbons (the carbons of Het joining the adjacent substituents) form a C5 to C7 cycloalkyl ring having up to double bonds including the double bond of the Het group. In other embodiments a cycloalkyl ring is present when R^{e} is a C3 to C8 cycloalkyl ring. The cycloalkyl groups can be substituted by one or more of the group consisting of alkyl-, alkoxycarbonyl-, amino-, aminocarbonyl-, carboxy-, fluoro-, or oxo- substituents. One of ordinary skill in the art will recognize that where cycloalkyl groups contain double bonds, the Sp² hybridized carbon atoms can contain only one substituent (which cannot be amino- or oxo-). Sp³ hybridized carbon atoms in the cycloalkyl ring can be geminally substituted with the exception that (1) two amino groups and (2) one amino and one fluoro group can not be substituted on the same Sp³ hybridized carbon atom.

In certain embodiments of the invention Het can be substituted on ring nitrogen atoms with hydrogen, alkyl, alkoxycarbonylalkyl-, Ar*, Ar*alkyl-, Ar*C(O)alkyl-, ArS*(O)alkyl-, Ar*S(O)₂alkyl-, so long as the ring nitrogen atoms are not “quaternized”. “Quaternized” herein is defined as a non-titrating salt, not including highly polar charge-neutralized covalent N-oxide bonds. By way of example a 1-alkylpyrazole is included in the invention. The ring nitrogen atom of the pyrazole to which the alkyl group is attached is referred to as not quaternized.

In certain embodiments of the invention, Het can be substituted on ring nitrogen with oxido (—O⁻). In these cases the ring N and the oxido group form an N-oxide.

In some embodiments of the invention, Y can be an oxo or mercapto group. Those of ordinary skill in the art will realize that different tautomeric forms for both of these groups can exist. The invention therefor contemplates the possibility of Y as hydroxy and Y as thioxo (═S).

In other compounds of the invention Y can be —NHC(O)(CH₂)ₙ—D—R^{e}R^{f}, wherein R^{e} is a group of the formula Het^{δ}. Het^{δ} can be attached from D to any site on Het^{δ}, so long as a stable chemical bond is formed. Preferably Het⁶⁷ is substituted onto D as Het is substituted onto Y.

Aryl or Ar* in addition to any substitutions specifically noted can be substituted with one or more substituents selected from the group of acylamino, acyloxyalkyl, alkanoyl, alkanoylalkyl, alkenyl, alkoxy, alkoxycarbonyl, alkoxycarbonylalkyl, alkyl, alkylamino, (C1-C3)alkylenedioxy, alkylsulfonyl [alkylS(O)₂—], alkylsulfinyl [alkylS(O)—], ω-alkylenesulfonic acid [-alkylSO₃H where n=1-6], alkylthio, allyl, amino, Ar*C(O)—, carboxy, carboxyalkyl, cycloalkyl, dialkylamino, halo, trifluoromethyl, hydroxy, (C2-C6)hydroxyalkyl, mercapto, nitro, morpholin-4-yl, thiomorpholin-4-yl, piperidin-1-yl, Ar*O—, Ar*-, Ar*-alkyl-, sulfamoyl, sulfonic acid [—SO₃H], 1-pyrrolidinyl, 4-[C6 or C10]arylpiperidin-1-yl and 4-[C6 or C10]arylpiperazin-1-yl.

Heterocycles except those of Het or Ar*, can be substituted with, in addition to substitutions specifically noted, one or more substituents selected from acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*-, Ar*-alkyl, carboxy, dialkylamino, fluoro, fluoroalkyl, difluoroalkyl, hydroxy, mercapto, 4-[C6 or C10]arylpiperidin-1-yl, 4-[C6 or C10]arylpiperazin-1-yl, (C1-C3)alkylenedioxy, oxo, sulfamoyl, and trifluoromethyl. Preferably multiple substituents are located on different atoms of the heterocyclic ring, with the proviso that alkyl, alkoxycarbonyl, and fluoro substituents can be substituted on the same carbon atom of the heterocyclic ring.

The halo atoms can be fluoro, chloro, bromo or iodo. Chloro and fluoro are preferred substituents for aryl substitutions.

In certain embodiments of this invention, the compounds of formula (I) can form biologically and pharmaceutically acceptable salts. Useful salt forms include the halides (particularly bromides and chlorides), tosylates, methanesulfonates, brosylates, fumarates, maleates, succinates, acetates, mesitylenesulfonates, and the like. Other related salts can be formed using similarly non-toxic, and biologically and pharmaceutically acceptable anions.

Where one or more compounds of formula I are administered to decrease intraocular pressure, at least one compound of formula I administered in effective amount is not a triazole, thiadiazole, tetrazole or pyridotriazole substituted on a ring carbon sulfonamide (the amide of which can be substituted) that has carbonic anhydrase inhibiting activity. Of course the composition can include an effective amount of a first agent, as well as a carbonic anhydrase-inhibiting effective amount of another agent, including one of those distinguished above.

Compounds of the general formula Het-Y can be prepared either by chemical syntheses well known in the art or by the methods described below. In addition, certain of the heterocycles useful as intermediates for the preparation of compounds of the invention are well-known and readily available from chemical supply houses or can be prepared by synthetic schemes specifically published therefor. The chemical reagents shown in the schemes below provide nonlimiting examples of means well known the art to carry out the reaction steps shown below.

As is recognized many of the nitrogen containing heterocycles of the invention are commercially available from chemical supply houses or are readily synthesized by methods well known in the art. For instance, certain substitution patterns can be obtained by electrophilic and nucleophilic substitution reactions on the heterocycle and are well known in the art. In addition selected nitrogen heterocycles are susceptible to metallation with organoalkali reagents, for example, n-butyllithium. The intermediate metallated-heterocycles can be treated with electrophiles to provide additional routes to substituted aromatic nitrogen heterocycles.

Certain aromatic nitrogen containing heterocycles can be obtained by cyclization and cycloaddition reactions of substituted acyclic precursors that are well known in the art. Nonlimiting examples of such syntheses are described below.

The pyrazole compounds of the invention can be prepared by reaction of hydrazine derivatives with 1,3-dicarbonyl compound (Scheme 1). For example, 1,3-dicarbonyl ketones having aryl substituents can be used to prepare 3-arylpyrazole (i.e. Y═Ar*) compounds. As will be recognized by those in the art, use of unsymmetrically substituted 1,3-dicarbonyl compounds with alkyl or aryl hydrazines often lead to isomeric mixtures of pyrazole products. These isomeric mixtures can be separated by well-known separation techniques such as fractional crystallization, column chromatography, and the like.

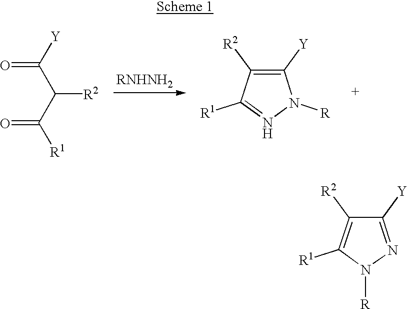

3-Aminopyrazole compounds (Y═NH₂) of the invention can be prepared by reaction of aryl hydrazones with ketones and aldehyde containing an α-nitrile moiety (Scheme 2, Bouveault, M. L. Bull. Soc. Chim. Fr., 1890, 4, 647). 3-Aminopyrazoles can also serve as intermediates for 3-acylamino-, 3-ureido-, and 3-thioureidopyrazoles of the invention. For example, 3-aminopyrazoles can be heated with esters to form 3-acylaminopyrazoles of the invention. The 3-aminopyrazoles are heated with formic acid to provide 3-formylaminopyrazoles. Likewise, treatment of 3-aminopyrazoles with isocyanates and isothiocyanates lead to the 3-ureido and 3-thioureido compounds (respectively) of the invention.

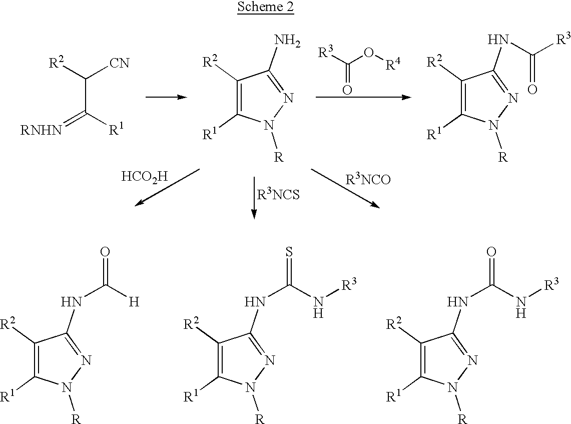

Indazoles of the invention substituted with alkyl and aryl substituents at the 3-position are synthesized from benzene analogs containing ortho-halo ketones and aldehydes (Scheme 3). For example, an indazole containing a 3-phenyl substituent can be prepared from a benzophenone analog containing a bromo moiety ortho to the carbonyl.

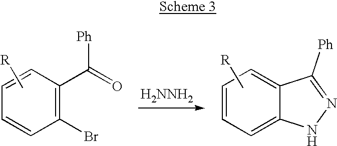

3-aminoindazoles are similarly prepared from substituted benzene precursors. A 2-azidobenzonitrile can be treated with hydrazine to prepared 3-aminoindazoles of the invention (Scheme 4, Paterson, T. M.; Smalley, R. K.; Sushizky Tetrahedron Lett., 1977, 3973). 3-Acylamino-, 3-ureido-, and 3-thioureidoindazoles of the invention can be prepared from the 3-aminoindazoles using esters, isocyanates, and isothiocyanates (as described above using 3-aminopyrazoles).

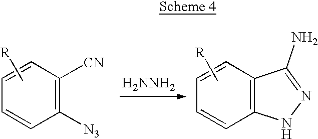

3- and 5-Aryl and alkyl isoxazoles of the invention are prepared by use of the chloro substituted α,β-unsaturated ketones with hydroxylamine (Scheme 5). The isomeric products can be isolated by separation techniques such as fractional crystallization, distillation, or column chromatography. Alternatively, 5-aryl substituted isoxazoles can be prepared from acetophenones (Scheme 5, Lin, Y. Lang, S. A. J. Heterocyclic Chem., 1977, 14, 355).

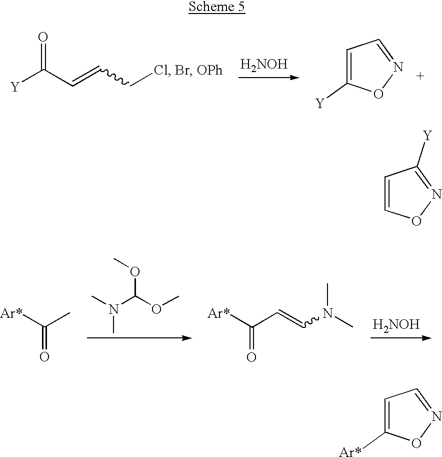

5-Aminoisoxazoles of the invention can be prepared from α-halo substituted oximes by reaction with sodium cyanide (Scheme 6, Lozanovic, M. et al. Chem. Abstr., 1981, 94, 192202c). The 5-amino group can be reacted with the reagents described above for 3-aminopyrazoles to provide acylamino-, ureido-, and thioureido isoxazoles of the invention.

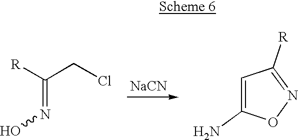

Alkyl and aryl substituted isothiazoles of the invention are prepared by the cyclization of β-imino thionocarbonyl compounds (Scheme 7). The cyclization is effected by oxidizing reagents well known in the art such as peroxides, chloranil, iodine, and the like. For example, starting material with an aryl thionocarbonyl group β-substituted to an imino group can be used to prepare a 5-aryl substituted isothiazole.

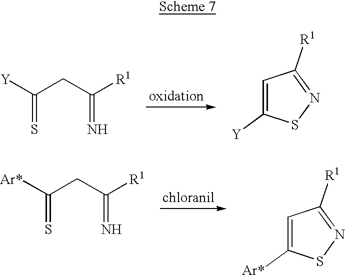

5-Amino isothiazoles of the invention can be prepared similarly (Scheme 8). Enamines can be treated with isothiocyanates to yield thioamide intermediates. The thioamides can be cyclized using oxidizing agents to provide 5-aminoisothiazoles of the invention. The 5-amino group can be reacted with the reagents described above for the 3-aminopyrazoles to provide acylamino-, formylamino-, ureido-, and thioureido-isoxazoles of the invention.

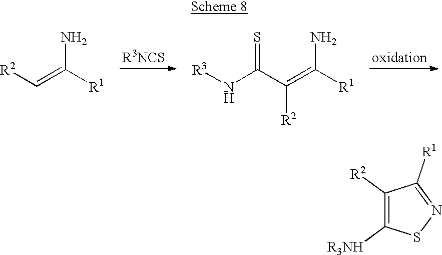

Aryl and alkyl 1,2,4-triazoles of the invention are prepared from acyl amidrazones as shown in Scheme 9. Amino-substituted 1,2,4-triazoles are formed analogously from acylaminoguanidine precursors.

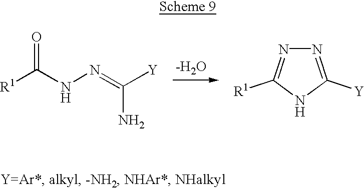

Many of the pyridine compounds of the invention can be obtained by methods familiar to those of ordinary skill in the art. For example, 2-aminopyridines of the invention are prepared by the Chichibabin reaction of pyridines with sodamide at temperatures of 100-120° C. (J. Russ. Phys. Chem. Soc., 1914, 46, 1216; Scheme 10). Alternatively 2-alkylamino or arylamino pyridines can be prepared by nucleophilic displacement of 2-chloropyridines, or preferably 2-fluoropyridines with amines. The amines can be acylated with alkanoyl or aroyl chlorides to provide acylamino compounds of the invention. The exocyclic amines can be heated with formic acid to provide formylamino pyridine compounds of the invention.

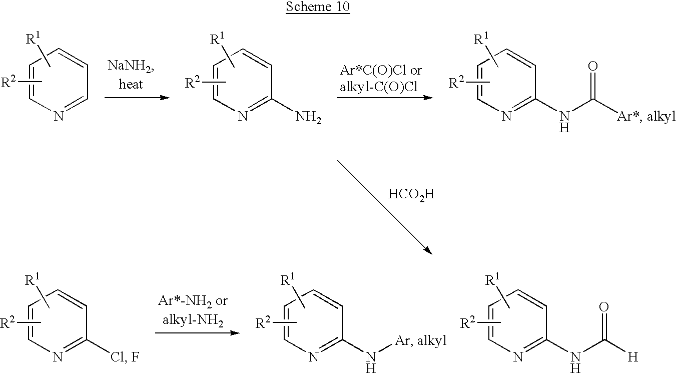

Quinolines of the invention can be obtained from substituted benzene precursors by a number of methods known to those of ordinary skill in the art. For example, variations of the Skraup synthesis of quinolines can be used as shown in Scheme 11 (Jones, G., Quinolines, Wiley-Interscience, New York, 1977, p 93).

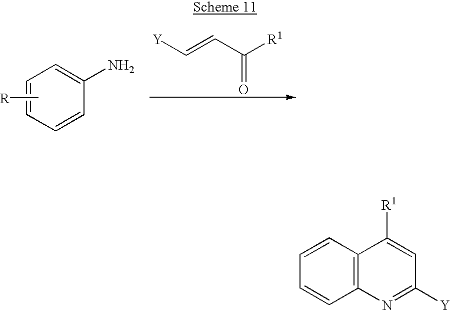

Substituted isoquinoline compounds of the invention can be prepared by Bischler-Napieralski reaction followed by an oxidation step (Scheme 12).

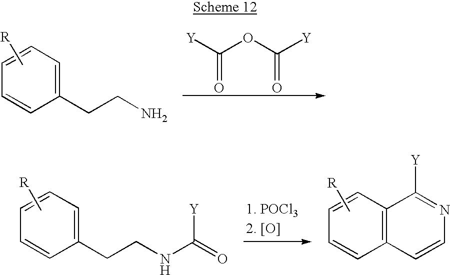

The preparation of 2-aminoquinolines and 1-aminoisoquinolines of the invention is analogous to the preparation of the pyridines of the invention. The amines can be acylated with alkanoyl and aroyl chlorides to provide acylamino compounds of the invention. The exocyclic amines can be heated with formic acid to provide formylamino pyridine compounds of the invention.

Pyridazine compounds of the invention can be prepared by reaction of hydrazine with 1,4-dicarbonyl compounds wherein R¹ and R² are alkyl or Ar* substituents. The dihydro pyridazines can be oxidized by, for example, air to give pyridazines (Scheme 13). Phthalazines can be prepared in analogous fashion.

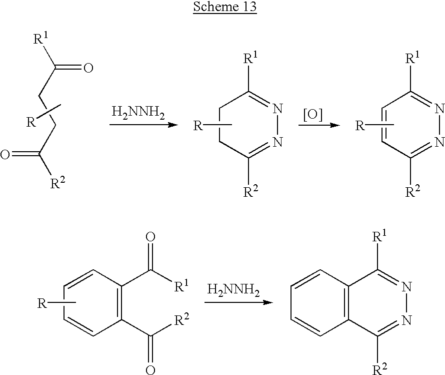

3-Aminopyridazines and 1-aminophthalazine (i.e. Y is —NH₂) can be prepared from pyridazines by analogous procedures to the preparation of 2-aminopyridine by heating with alkali amides (Scheme 14). Aminopyridazines can also be prepared from the reaction of hydrazine with acrylonitriles containing β-carbonyl groups by heating in a solvent such as ethanol. Alternatively 3-aminopyriydazines can be prepared from 3-fluoro substituted pyridazines by displacement of the fluoro group by amines such as alkylamines and arylamines.

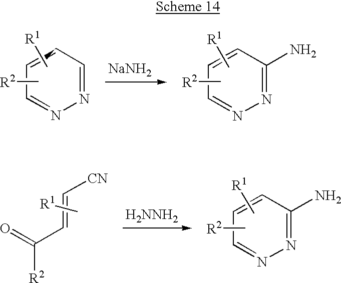

Benzonitriles with ortho-ketones or aldehydes are reacted with hydrazine to prepare 1-aminophthalazines of the invention (Scheme 15).

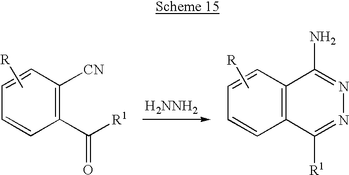

Cinnolines of the invention are prepared by cyclization of diazonium salts containing an ortho vinyl group (Scheme 16).

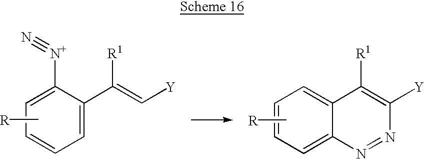

Substituted pyrimidines can be obtained, for example, by the condensation of alkyl and aryl amidines with 1,3-dicarbonyl compounds (Scheme 17) or α,β-unsaturated carbonyl compounds such as 3-ethoxymethacrolein. Use of alkylamidines in the condensation provides compounds of the invention, wherein Y is alkyl. The use of Ar* substituted amidines provide compounds wherein Y is Ar*. Finally, use of guanidines in the condensation provide compounds wherein Y is amino or substituted amino. For example, condensation of substituted acetylacetone analogs with guanidine or acetamidine yields substituted 2-amino and 2-methylpyrimidines, respectively (Bell, S. C.; Caldwell, W. T. J. Org. Chem., 1961, 26, 3534).

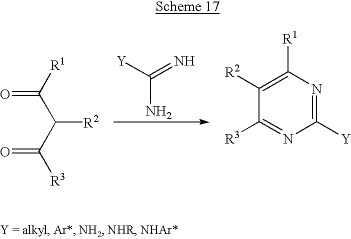

Benzo-fused pyrimidines (i.e., quinazolines) can be prepared from benzene analogs containing amino substituent ortho to a carbonyl (ketone or aldehyde) by acylation of the amino group with an alkanoyl or aroyl group, followed by cyclization of the acylamino intermediate with ammonia (Scheme 18).

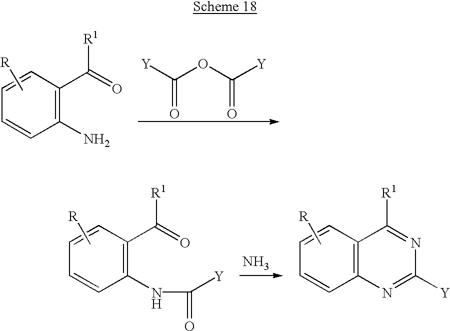

Quinazolines of the invention, wherein Y is an amino or substituted amino group, can be prepared by the sequence shown in Scheme 19. An aniline having an orthocarbonyl (or nitrile) substituent is treated with methyl chloroformate to provide a ureido substituted benzene intermediate. This intermediate can be cyclized with ammonia to provide a 2-oxoquinazoline intermediate. The 2-oxo group can be converted to a 2-chloro group with phosphorus oxychloride (POC₁₃). Displacement of the chloro group with amines provides 2-amino substituted quinazolines. Examples of the preparation of quinazolines of this type are described in U.S. Pat. No. 4,672,116.

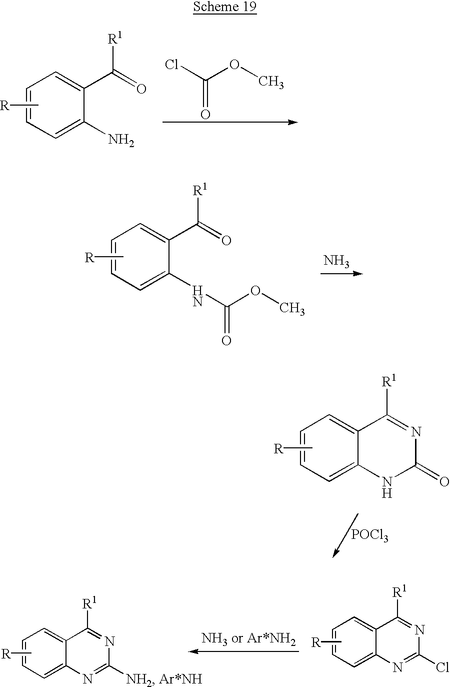

The amino groups of the amino-pyrimidines and -quinazolines are acylated with acid chlorides or anhydrides to form acylamino compounds of the inventions. Similarly the amino groups of the amino-pyrimidines and -quinazolines can be formylated with formic acid to provide formylamino compounds of the invention.

In addition to being useful for the methods of the instant invention, the nitrogen containing aromatic heterocycles of the invention can be used as suitable synthetic intermediates for the preparation of N-alkylated and aminated positively charged nitrogen heterocycles which are useful for treating the indications discussed herein. Methods for the synthesis and use of positively charged nitrogen heterocycles of this type are described in the provisional application entitled “Methods for the Treatment of Glaucoma III” (attorney docket number 361331-508P) filed Dec. 29, 2000, the disclosure of which is are herein incorporated by reference in its entirety.

To treat glaucoma or reduced accommodation and its associated symptoms, an effective amount of a pharmaceutical compound will be recognized by clinicians but includes an amount effective to treat, reduce, ameliorate, eliminate or prevent one or more symptoms of the disease sought to be treated or the condition sought to be avoided or treated, or to otherwise produce a clinically recognizable change in the pathology of the disease or condition.

In treating glaucoma, agents of the inventions can be administered concurrently or in a combined formulation with one or more α₂-selective adrenergic agonists, carbonic anhydrase inhibitors or prostaglandin analogs. Examples of α₂-selective adrenergic agonists include clonidine, apraclonidine, guanfacine, guanabenz and methyldopa, which are administered in effective amounts as is known in the art. Examples of carbonic anhydrase inhibitors include acetazolamide, dichlorphenamide and methazolamide, which are administered in effective amounts as is known in the art. Examples of prostaglandin analogs include PGE₂ and PGF_{2α} analogs, which are administered in effective amounts as is known in the art, including effective amounts administered by topical application to the eye. Thus, the invention further provides pharmaceutical compositions comprising an agent of the invention in combination with an effective amount of an α2-selective adrenergic agonist, carbonic anhydrase inhibitor, prostaglandin analog, or combination thereof.

Pharmaceutical compositions can be prepared to allow a therapeutically effective quantity of the compound of the present invention, and can include a pharmaceutically acceptable carrier, selected from known materials utilized for this purpose. See, e.g., Remington, The Science and Practice of Pharmacy, 1995; Handbook of Pharmaceutical Excipients, 3^{rd} Edition, 1999. Such compositions can be prepared in a variety of forms, depending on the method of administration.

In addition to the subject compound, the compositions of this invention can contain a pharmaceutically-acceptable carrier. The term “pharmaceutically-acceptable carrier”, as used herein, means one or more compatible solid or liquid filler diluents or encapsulating substances that are suitable for administration to an animal, including a mammal or human. The term “compatible”, as used herein, means that the components of the composition are capable of being commingled with the subject compound, and with each other, such that there is no interaction which would substantially reduce the pharmaceutical efficacy of the composition under ordinary use. Preferably when liquid dose forms are used, the compounds of the invention are soluble in the components of the composition. Pharmaceutically-acceptable carriers must, of course, be of sufficiently high purity and sufficiently low toxicity to render them suitable for administration to the animal being treated.

Some examples of substances which can serve as pharmaceutically-acceptable carriers or components thereof are sugars, such as lactose, glucose and sucrose; starches, such as corn starch and-potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject compound is basically determined by the way the compound is to be administered. If the subject compound is to be injected, the preferred pharmaceutically-acceptable carrier is sterile, physiological saline, with a blood-compatible suspending agent, the pH of which has been adjusted to about 7.4.

If the preferred mode of administering the subject compound is perorally, the preferred unit dosage form is therefore tablets, capsules, lozenges, chewable tablets, and the like. Such unit dosage forms comprise a safe and effective amount of the subject compound, which is preferably from about 0.7 or 3.5 mg to about 280 mg/ 70 kg, more preferably from about 0.5 or 10 mg to about 210 mg/ 70 kg. The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder-mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of this invention, and can be readily made by a person skilled in the art.

Peroral compositions also include liquid solutions, emulsions, suspensions, and the like. The pharmaceutically-acceptable carriers suitable for preparation of such compositions are well known in the art. Such liquid oral compositions preferably comprise from about 0.012% to about 0.933% of the subject compound, more preferably from about 0.033% to about 0.7%. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. Peroral liquid compositions may also contain one or more components such as sweeteners, flavoring agents and colorants disclosed above.

Other compositions useful for attaining systemic delivery of the subject compounds include sublingual and buccal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Compositions can also be used to deliver the compound to the site where activity is desired; such as eye drops, gels and creams for ocular disorders.

Compositions of this invention include solutions or emulsions, preferably aqueous solutions or emulsions comprising a safe and effective amount of a subject compound intended for topical intranasal administration. Such compositions preferably comprise from about 0.01% to about 10.0% w/v of a subject compound, more preferably from about 0.1% to about 2.0%. Similar compositions are preferred for systemic delivery of subject compounds by the intranasal route. Compositions intended to deliver the compound systemically by intranasal dosing preferably comprise similar amounts of a subject compound as are determined to be safe and effective by peroral or parenteral administration. Such compositions used for intranasal dosing also typically include safe and effective amounts of preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acids and bases to adjust the pH of these aqueous compositions as needed. The compositions may also comprise local anesthetics or other actives. These compositions can be used as sprays, mists, drops, and the like.

Other preferred compositions of this invention include aqueous solutions, suspensions, and dry powders comprising a safe and effective amount of a subject compound intended for atomization and inhalation administration. Such compositions are typically contained in a container with attached atomizing means. Such compositions also typically include propellants such as chlorofluorocarbons 12/11 and 12/114, and more environmentally friendly fluorocarbons, or other nontoxic volatiles; solvents such as water, glycerol and ethanol, these include cosolvents as needed to solvate or suspend the active; stabilizers such as ascorbic acid, sodium metabisulfite; preservatives such as cetylpyridinium chloride and benzalkonium chloride; tonicity adjustors such as sodium chloride; buffers; and flavoring agents such as sodium saccharin. Such compositions are useful for treating respiratory disorders, such as asthma and the like.

Other preferred compositions of this invention include aqueous solutions comprising a safe and effective amount of a subject compound intended for topical intraocular administration. Such compositions preferably comprise from about 0.01% to about 0.8% w/v of a subject compound, more preferably from about 0.05% to about 0.3%. Such compositions also typically include one or more of preservatives, such as benzalkonium chloride or thimerosal; vehicles, such as poloxamers, modified celluloses, povidone and purified water; tonicity adjustors, such as sodium chloride, mannitol and glycerin; buffers such as acetate, citrate, phosphate and borate; antioxidants such as sodium metabisulfite, butylated hydroxy toluene and acetyl cysteine; acids and bases can be used to adjust the pH of these formulations as needed.

Other preferred compositions of this invention useful for peroral administration include solids, such as tablets and capsules, and liquids, such as solutions, suspensions and emulsions (preferably in soft gelatin capsules), comprising a safe and effective amount of a subject compound. Such compositions can be coated by conventional methods, typically with pH or time-dependent coatings, such that the subject compound is released in the gastrointestinal tract at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit™ coatings, waxes and shellac.

The compounds of the invention are administered by ocular, oral, parenteral, including, for example, using formulations suitable as eye drops. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. See, Remington's Pharmaceutical Sciences, 16th Ed., Mack Publishing, Easton, Pa., 1980, as well as later editions, for information on pharmaceutical compounding.

Numerous additional administration vehicles will be apparent to those of ordinary skill in the art, including without limitation slow release formulations, liposomal formulations and polymeric matrices.

In another preferred embodiment, the pharmaceutically effective amount is approximately 0.1 or 0.5 to 4 mg/kg body weight daily. Still more preferably, the pharmaceutically effective amount is approximately 1 mg/kg body weight daily. In a preferred embodiment, the amount is administered in once daily doses, each dose being approximately 1 mg/kg body weight.

Compounds of the invention can be used in conjunction with monitoring the improvement (decrease) in the intraocular pressure in a mammal using standard methodology.

The methods of the inventions can be assessed in animal models for ophthalmologic function. For example, improvements in fluid outflow facility can be studied in Rhesus monkeys treated with the compounds and methods of the invention. Aged Rhesus monkeys receive a single transcorneal injection of a test compound (compound of the invention) at a concentration of about 1 mM in the anterior chamber of one eye, and Barany's solution, as a control, in the adjacent eye. Needle outflow facility is measured under baseline and pilocarpine-stimulated conditions at time points (for example, 3, 8, 12 and 24 weeks), after the administration of the test compound. Increases in outflow facility in the drug treated vs. the control eye under baseline and cholinergic-stimulated (e.g. pilocarpine) conditions at the various time points are compared. As the enhancement of outflow facility can be influenced by the route of administration of the cholinergic agent, various routes of administration of the cholinergic agent can be used in the experiments. For instance, an intravenous administration versus a direct administration of pilocarpine can be compared. The above experiment demonstrates one method of measuring the improvement in ophthalmologic function. Such improvement has been illustrated with 4,5-dimethyl-3-(2-oxoethyl-phenethyl)thiazolium chloride, a compound believed to act by the same mechanism as those described here. See, U.S. application for “Methods for Treating Glaucoma I,” concurrently filed herewith.

In addition to measuring increased fluid outflow facility using the methods of the invention, improvements in pilocarpine-stimulated accommodation (i.e., the process of effecting refractive changes in the shape of the lens) can also be assessed in animal studies. As in the regulation of outflow facility, cholinergic input stimulates the movement of the ciliary muscle to control the shape of the lens, and allows accommodation in conditions of low illumination. Accommodation is impaired in a vast majority of individuals and begins to become noticeable to the individual around the age of 40 years. Interestingly, changes in accommodative response occur much earlier in life, around 18 years of age, and progresses until vision is noticeably impaired.

Physiological studies on accommodation are conducted following intraocular injection of a test compound and the results are compared relative to the results of control (untreated) animals. In the experiment, primates(for example, Rhesus monkeys) are treated twice a day for four days with 2 μg of prostaglandin F2α (PGF2α). On days 5-8 both eyes are treated first with 2 μg of PGF2α followed 2 hours later with an intraocular injection of 10 μL of the test compound of a final concentration of 1 mM. No injection is made to the control eye. 24 Hours after the last injection of the test compound, a course of therapy consisting of once a day dosing for a total of 4 days accommodative responses to i.m. pilocarpine administration is performed following phenylephrine refraction. Improvement in accommodation has been illustrated with 4,5-dimethyl-3-(2-oxoethyl-phenethyl)thiazolium chloride, a compound believed to act by the same mechanism as those described here. See, U.S. application for “Methods for Treating Glaucoma I,” concurrently filed herewith.

Compounds of the invention can be tested to determine corneal penetration to the anterior chamber of the eye following topical administration of eye drops. For example, a test compound is assayed in vitro through an intact rabbit cornea for transcorneal penetration in a standard diffusion chamber apparatus. Corneas are mounted in a chamber at 37° C. with the epithelial side exposed to the test compound in Barany's solution. 1.0 mL samples are taken from the endothelial side 1 hour after addition of the test compound at a final concentration of 1 mM to the epithelial chamber. The volume of the chamber is replaced with phosphate buffered saline. The amount of test compound can be measured using any means that can be used to separate the compound and measure its concentration. For example, an HPLC with an attached UV detector can be used to determine the concentration of the test compound that has penetrated the cornea. Penetration values are also determined at later time points, for example, at 5 hours.

Assessment of corneal penetration of compounds of the invention can be determined in vivo, for example, in Cynomolgus monkeys. During these studies, the penetration of a test compound is evaluated using an eye-cup which holds a solution of 10 mM of the test compound in Barany's solution for 5 hours. At the end of the experiment the eye cup is removed, the eye is repeatedly flooded with Barany's solution and a sample of intraocular fluid is removed from the anterior chamber with a needle inserted through the cornea. The quantity of the test compound in the intraocular fluid is determined using, for example, HPLC methods.

The activity of the compounds of the invention in breaking, reversing or inhibiting the formation of AGE's or AGE-mediated crosslinks can be assayed by any of the methods described in U.S. Pat. No. 5,853,703.

### EXAMPLE 1

### Cross-Linking Inhibition Assay

The following method was used to evaluate the ability of the compounds to inhibit the cross-linking of glycated bovine serum albumin (AGE-BSA) to rat tail tendon collagen-coated 96-well plates.

AGE-BSA was prepared by incubating BSA at a concentration of 200 mg per ml with 200 mM glucose in 0.4M sodium phosphate buffer, pH 7.4 at 37° C. for 12 weeks. The glycated BSA was then extensively dialyzed against phosphate buffer solution (PBS) for 48 hours with additional 5 times buffer exchanges. The rat tail tendon collagen coated plate was blocked first with 300 microliters of Superbloc blocking buffer (Pierce Chemical, Rockford, Ill.) for one hour. The blocking solution was removed from the wells by washing the plate twice with phosphate buffered saline (PBS)-Tween 20 solution (0.05% Tween 20) using a NUNC-multiprobe (Nalge Nunc, Rochester, N.Y.) or Dynatech ELISA-plate (Dynatech, Alexandria, Va.) washer. Cross-linking of AGE-BSA (1 to 10 microgram per well depending on the batch of AGE-BSA) to rat tail tendon collagen coated plate was performed with and without the testing compound dissolved in PBS buffer at pH 7.4 at one or more desired concentrations by the addition of 50 microliters each of the AGE-BSA diluted in PBS or in the solution of test compound at 37° C. for 4 hours. Unbrowned BSA in PBS buffer with or without testing compound were added to the separate wells as the blanks. The un-cross-linked AGE-BSA was then removed by washing the wells three times with PBS-Tween buffer. The amount of AGE-BSA crosslinked to the tail tendon collagen-coated plate was then quantitated using a polyclonal antibody raised against AGE-RNase. After a one-hour incubation period, AGE antibody was removed by washing 4 times with PBS-Tween.

The bound AGE antibody was then detected with the addition of horseradish peroxidase-conjugated secondary antibody—goat anti-rabbit immunoglobulin and incubation for 30 minutes. The substrate of 2,2-azino-di(3-ethylbenzthiazoline sulfonic acid) (ABTS chromogen) (Zymed Laboratories, Inc., South San Francisco, Calif.) was added. The reaction was allowed for an additional 15 minutes and the absorbance was read at 410 nm in a Dynatech plate reader.

### EXAMPLE 2

### Cross-Link Breaking Assay

To ascertain the ability of the compounds of the instant invention to break or reverse already formed advanced glycosylation endproducts, a sandwich enzyme immunoassay was applied. Generally, the assay utilizes collagen-coated 96 well microtiter plates that are obtained commercially. AGE-modified protein (AGE-BSA) is incubated on the collagen-coated wells for four hours, is washed off the wells with PBS-Tween and solutions of the test compounds are added. Following an incubation period of 16 hours (37° C.) cross-link-breaking is detected using an antibody raised against AGE-ribonuclease or with an antibody against BSA.

### Preparation of Solutions and Buffers

Bovine Serum Albumin (Type V) (BSA) (from Calbiochem) solution was prepared as follows: 400 mg of Type V BSA (bovine serum albumin) was added for each ml of 0.4 M sodium phosphate buffer, pH 7.4. A 400 mM glucose solution was prepared by dissolving 7.2 grams of dextrose in 100 ml of 0.4 M sodium phosphate buffer, pH 7.4. The BSA and glucose solutions were mixed 1:1 and incubated at 37° C. for 12 weeks. The pH of the incubation mixture was monitored weekly and adjusted to pH 7.4 if necessary. After 12 weeks, the AGE-BSA solution was dialyzed against PBS for 48 hours with four buffer changes, each at a 1:500 ratio of solution to dialysis buffer. Protein concentration was determined by the micro-Lowry method. The AGE-BSA stock solution was aliquoted and stored at −20° C.

Test compounds were dissolved in PBS and the pH was adjusted to pH 7.4, if necessary. AGE-BSA stock solution was diluted in PBS to measure maximum crosslinking and in the inhibitor solution for testing inhibitory activity of compounds. The concentration of AGE-BSA necessary to achieve the optimum sensitivity was determined by initial titration of each lot of AGE-BSA.

Substrates for detection of secondary antibody binding were prepared by diluting the HRP substrate buffer (Zymed) 1:10 in distilled water and mixing with ABTS chromogen (Zymed) 1:50 just prior to use.

### Assay Procedures

Biocoat plates were blocked with 300 microliters of Superbloc (Pierce Chemical). Plates were blocked for one hour at room temperature and were washed with PBS-Tween (0.05% v/v) three times with the Dynatech platewasher before addition of test reagents.

The first three wells of the Biocoat plate were used for the reagent blank. Fifty microliters of solutions AGE-BSA were added to test wells in triplicate and only PBS in blank wells. The plate was incubated at 37° C. for four hours and washed with PBS-Tween three times. Fifty microliters of PBS was added to the control wells and 50 microliters of the test prospective agent was added to the test wells and blank. The plate was incubated overnight (approximately 16 hours) with prospective agent, followed by washing in PBS before addition of primary antibody.

(Prior to use, each lot of primary antibody, either anti-BSA or anti-RNase, was tested for optimum binding capacity in this assay by preparing serial dilutions (1:500 to 1:2000) and plating 50 microliters of each dilution in the wells of Biocoat plates. Optimum primary antibody was determined from saturation kinetics.) Fifty microliters of primary antibody of appropriate dilution, was added and incubated for one hour at room temperature. The plate was then washed with PBS-Tween.

Plates were incubated with the secondary antibody, HRP-(Goat-anti-rabbit), which was diluted 1:4000 in PBS and used as the final secondary antibody. The incubation was performed at room temperature for thirty minutes.

Detection of maximum crosslinking and breaking of AGE crosslinking was performed as follows. HRP substrate (100 microliter) was added to each well of the plate and was incubated at 37° C. for fifteen minutes. Readings were taken in the Dynatech ELISA-plate reader.

Except where heteroaryl is separately recited for the same substituent, the term “heterocycle” includes heteroaryl.

Where noted above, publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of decreasing intraocular pressure or improving ocular accommodation in an animal, including a human, comprising administering an intraocular pressure decreasing or ocular accommodation improving amount of a compound of the formula I:
Het-Y (I)
wherein:
a. Het is a five or six membered heterocycle having a first ring nitrogen and optionally, a second or third ring nitrogen, with the remaining ring atoms being carbon, oxygen, or sulfur; provided that Het is not thiazole, imidazole, oxazole, or dihydro or tetrahydro analogs thereof,
b. Het can be substituted on carbon atoms with
1. one or more substituents independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl (which alkyl can be substituted with alkyloxyimino), cycloalkyl, dialkylamino, halo, hydroxy, (C₂-C₆)hydroxyalkyl, mercapto, nitro, sulfamoyl, sulfonic acid, alkylthio, alkylsulfonyl, alkylsulfinyl, alkylsulfonamido, trifluoromethyl, morpholin-4-yl, 4-[C₆ or C₁₀]arylpiperidin-1-yl, 4-[C₆ or C₁₀]arylpiperazin-1-yl, thiomorpholin-4-yl, piperidin-1-yl, Ar* {wherein, consistent with the rules of aromaticity, Ar* is C₆ or C₁₀ aryl or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring can be optionally fused to a substituted benzene, pyridine, pyrimidine, pyridazine, pyrazine, or (1,2,3)triazine (wherein the ring fusion is at a carbon-carbon double bond of Het)}, Ar*-alkyl, Ar*-O, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or
2. two adjacent substitutions together with their ring carbons form a fused C₆ or C₁₀ aryl ring which aryl ring can be substituted as set forth below; or
3. two adjacent substitutions together with their ring carbons form a C₅-C₇ fused cycloalkyl ring having up to two double bonds including any fused double bond of the Het group, which cycloalkyl ring can be substituted by one or more of the group consisting of alkyl, alkoxycarbonyl, amino, aminocarbonyl, carboxy, fluoro, or oxo; or
4. two adjacent substitutions together with their ring carbons form a fused 5- or 6-membered heteroaryl ring, wherein the 6-membered heteroaryl ring contains one to three atoms of N, and The 5-membered heteroaryl ring contains from one to three atoms of N or one atom of O or S and zero to two atoms of N; or
5. two adjacent substitutions together with their ring carbons form a fused five to eight membered fused heterocycle, wherein the ring fusion is at a carbon-carbon bond of Het, wherein the fused heterocycle consists of ring atoms selected from the group consisting of carbon, nitrogen, oxygen, sulfur, or S(O)ₙ, wherein S(O)n is 1 or 2; and
c. Het can be substituted on ring nitrogen atoms with
1. hydrogen, alkyl, alkoxycarbonylalkyl-, Ar*, Ar*alkyl-, Ar*C(O)alkyl-, ArS*(O)alkyl-, Ar*S(O)₂alkyl-, so long as the ring nitrogen atoms are not quaternized;
2. amino; or
3. at most one nitrogen with oxido (—O⁻) to form an N-oxide; and
d. Y is substituted on a ring carbon adjacent to the first or second ring nitrogens and is
1. hydrogen, oxo, alkyl, mercapto, alkylthio, amino, amino(C₁-C₅)alkyl, or aminophenyl, wherein the amino of the latter three groups can be (a) substituted with
(a) Ar*,
(b) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂ or
(c) formyl or alkanoyl,
2. —NHC(O)(CH₂)ₙ—D—R^{e}R^{f}, wherein D is oxygen, sulfur or nitrogen, wherein when D is nitrogen n is 0, 1 or 2, but when D is oxygen or sulfur n=1 or 2, and R^{f} is present only when D is nitrogen, wherein
(a) R^{e} is
(1) Ar*, or
(2) a group of the formula
Het^{δ}-
wherein Het^{δ} is independently the same as Het, or
(3) a C₃-C₈ cycloalkyl ring having up to one double bond with the proviso that the carbon linking the cycloalkyl ring to D is saturated, which cycloalkyl ring can be substituted by one or more alkyl-, alkoxycarbonyl-, amino-, aminocarbonyl-, carboxy-, fluoro-, or oxo-substituents, or
(4) hydrogen, (C₂-C₆)hydroxyalkyl, alkanoylalkyl, alkyl, alkoxycarbonylalkyl, alkenyl, carboxyalkyl (which alkyl can be substituted with alkoxyimino), alkoxycarbonyl, Ar*, or Ar*-alkyl-; and
(b) R^{f} is independently hydrogen, hydroxy(C₂-C₆)alkyl, alkanoylalkyl, alkyl, alkoxycarbonylalkyl, alkenyl, carboxyalkyl (which alkyl can be substituted with alkyloxyimino), alkoxycarbonyl, independently a group Ar* or Ar*-alkyl;
wherein aryl or Ar* in addition to any substitutions specifically noted can be substituted with one or more substituents selected from the group of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*C(O)NH—, carboxy, carboxyalkyl, cycloalkyl, dialkylamino, halo, trifluoromethyl, hydroxy, (C₂-C₆)hydroxyalkyl, mercapto, nitro, morpholin-4-yl, thiomorpholin-4-yl, piperidin-1-yl, Ar*O—, Ar*-, Ar*-alkyl-, sulfamoyl, sulfonic acid, 1-pyrrolidinyl, piperidin-1-yl, 4-[C₆ or C₁₀]arylpiperidin-1-yl, and 4-[C₆ or C₁₀]arylpiperazin-1-yl; and
heterocycles except those of Het or Ar*, can be substituted with, in addition to substitutions specifically noted, one or more substituents selected from acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*-, Ar*-alkyl, carboxy, dialkylamino, fluoro, fluoroalkyl, difluoroalkyl, hydroxy, mercapto, 4-[C₆ or C₁₀]arylpiperidin-1-yl, 4-[C₆ or CIO]arylpiperazin-1-yl, (C₁-C₃)alkylenedioxy, oxo, sulfamoyl, and trifluoromethyl; or a pharmaceutically acceptable salt of said compounds,
with the proviso that where the compound of formula I is administered to decrease intraocular pressure at least one compound of formula I administered in effective amount is not a triazole, thiadiazole, tetrazole or pyridotriazole substituted on a ring carbon sulfonamide (the amide of which can be substituted) that has carbonic anhydrase inhibiting activity.

2. The method of claim 1, wherein
a. Het is a five or six membered heterocycle having a first ring nitrogen and optionally, a second or third ring nitrogen, with the remaining ring atoms being carbon, oxygen, or sulfur; provided that Het is not thiazole, imidazole, oxazole, or dihydro or tetrahydro analogs thereof;
b. Het can be substituted on carbon atoms with
1. one or more substituents independently selected from hydrogen, acylamino, alkanoyl, alkanoylalkyl, alkoxy, alkoxycarbonyl, alkoxycarbonylalkyl, alkyl, alkylamino, amino, ω-alkylenesulfonic acid, carbamoyl, carboxy, carboxyalkyl (which alkyl can be substituted with alkyloxyimino), cycloalkyl, dialkylamino, halo, hydroxy, (C₂-C₆)hydroxyalkyl, mercapto, nitro, sulfamoyl, sulfonic acid, alkylthio, alkylsulfonyl, alkylsulfinyl, alkylsulfonamido, trifluoromethyl, morpholin-4-yl, thiomorpholin-4-yl, 4-[C₆ or C₁₀]arylpiperidin-1-yl, 4-[C₆ or C₁₀]arylpiperazin-1-yl, piperidin-1-yl, Ar* {wherein, consistent with the rules of aromaticity, Ar* is C₆ or C₁₀ aryl or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring can be optionally fused to a substituted benzene, pyridine, pyrimidine, pyridazine, pyrazine, or (1,2,3)triazine (wherein the ring fusion is at a carbon-carbon double bond of Het)}, Ar*-alkyl, Ar*-O, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or
2. two adjacent substitutions together with their ring carbons form a fused C₆ or C₁₀ aryl ring which aryl ring can be substituted as set forth below; or
3. two adjacent substitutions together with their ring carbons form a C₅-C₇ fused cycloalkyl ring having no double bonds except any fused double bond of the Het group, which cycloalkyl ring can be substituted by one or more of the group consisting of alkyl, alkoxycarbonyl, amino, aminocarbonyl, carboxy, fluoro, or oxo; and
c. Het can be substituted on ring nitrogen atoms with
1. hydrogen, alkyl, alkoxycarbonylalkyl-, Ar*, Ar*alkyl-, Ar*C(O)alkyl-, ArS*(O)alkyl-, Ar*S(O)₂alkyl-, so long as the ring nitrogen atoms are not quaternized;
2. amino; or
3. at most one nitrogen with oxido (—O⁻) to form an N-oxide; and
d. Y is substituted on a ring carbon adjacent to the first or second ring nitrogens and is
1. hydrogen, oxo, alkyl, mercapto, alkylthio, amino, amino(C₁-C₅)alkyl, or aminophenyl, wherein the amino of the latter three groups can be (a) substituted with
(a) Ar*,
(b) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂ or
(c) formyl or alkanoyl,
2. —NHC(O)(CH₂)ₙ—D—R^{e}R^{f}, wherein D is oxygen, sulfur or nitrogen, wherein when D is nitrogen n is 0, 1 or 2, but when D is oxygen or sulfur n=1 or 2 and R^{f} is present only when D is nitrogen, wherein
(a) R^{e} is
(1) Ar*
(2) a group of the formula
Het^{δ}-
wherein Het^{δ} is independently the same as Het,
(3) a C₃-C₈ cycloalkyl ring having up to one double bond with the proviso that the carbon linking the cycloalkyl ring to D is saturated, which cycloalkyl ring can be substituted by one or more alkyl-, alkoxycarbonyl-, amino-, aminocarbonyl-, carboxy-, fluoro-, or oxo-substituents, where multiple substituents are located on different carbon atoms of the cycloalkyl ring, except in the case of alkyl, alkoxycarbonyl, and fluoro substituents, which can be located on the same or different carbon atoms; or
(4) hydrogen, (C₂-C₆)hydroxyalkyl, alkanoylalkyl, alkyl, alkoxycarbonylalkyl, alkenyl, carboxyalkyl (which alkyl can be substituted with alkoxyimino), alkoxycarbonyl, Ar*, or Ar*-alkyl-; and
(b) R^{f} is independently hydrogen, hydroxy(C₂-C₆)alkyl, alkanoylalkyl, alkyl, alkoxycarbonylalkyl, alkenyl, carboxyalkyl (which alkyl can be substituted with alkyloxyimino), alkoxycarbonyl, independently a group Ar* or Ar*-alkyl;
wherein aryl or Ar* in addition to any substitutions specifically noted can be substituted with one or more substituents selected from the group of alkyl, amino, dialkylamino, 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl; and
heterocycles except those of Het or Ar*, can be substituted with, in addition to substitutions specifically noted, one or more substituents selected from acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*-, Ar*-alkyl, carboxy, dialkylamino, fluoro, fluoroalkyl, difluoroalkyl, hydroxy, mercapto, 4-[C₆ or C₁₀]arylpiperidin-1-yl, 4-[C₆ or C₁₀]arylpiperazin-1-yl. (C₁-C₃)alkylenedioxy, oxo, sulfamoyl, and trifluoromethyl;
or a pharmaceutically acceptable salt of said compounds.

3. The method of claim 1, wherein Het-Y is 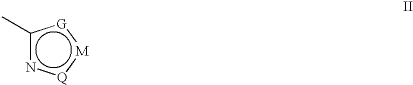
wherein G, M, and Q are selected from the group consisting of O, S, C—R^{h}, C—Rⁱ, and N—R^{g}, with the proviso that only one of G or Q can be O or S,
a. wherein R^{g}, is
(1) hydrogen, alkyl, alkoxycarbonylalkyl-, Ar*, Ar*-alkyl-, Ar*C(O)alkyl-, Ar*S(O)alkyl-, or Ar*S(O)₂alkyl-, so long as the ring nitrogen atoms are not quaternized; or
(2) amino or oxido (wherein N—R^{g} forms an N-oxide) and
b. wherein R^{h} or Rⁱ are
(1) independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl (which alkyl can be substituted with alkyloxyimino), cycloalkyl, dialkylamino, halo, hydroxy, (C₂-C₆)hydroxyalkyl, mercapto, nitro, sulfamoyl, sulfonic acid, alkylthio, alkylsulfonyl, alkylsulfinyl, alkylsulfonamido, trifluoromethyl, morpholin-4-yl, 4-[C₆ or C₁₀]arylpiperidin-1-yl, 4-[C₆ or C₁₀]arylpiperazin-1-yl, thiomorpholin-4-yl, piperidin-1-yl, Ar*, Ar*-alkyl, Ar*-O, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or;
(2) R^{h} and Rⁱ where adjacent, together with their ring carbons form a C₅-C₇ fused cycloalkyl ring having up to two double bonds including the fused double bond of the Het group, which cycloalkyl ring can be substituted by one or more of the group consisting of alkyl-, alkoxycarbonyl-, amino-, aminocarbonyl-, carboxy-, fluoro-, or oxo- substituents, except in the case of alkyl, alkoxycarbonyl, and fluoro substituents, which can be located on the same or different carbon atoms;
(3) R^{h} and Rⁱ where adjacent, together with their ring carbons form a fused C₆ or C₁₀ aryl ring;
(4) R^{h} and Rⁱ where adjacent, together with their ring carbons form a fused five to eight membered fused heterocycle, wherein the ring fusion is at a carbon-carbon bond of Het, wherein the fused heterocycle consists of ring atoms selected from the group consisting of carbon, nitrogen, oxygen, sulfur, or S(O)n, wherein S(O)ₙ is 1 or 2; or
(5) R^{h} and Rⁱ where adjacent, together with their ring carbons form a fused 5- or 6-membered heteroaryl ring containing at least one and up to three atoms of N for the 6-membered fused heteroaryl rings and from one to three atoms of N or one atom of O or S and zero to two atoms of N for the 5-membered fused heteroaryl rings.

4. The method of claim 3, wherein Het-Y is 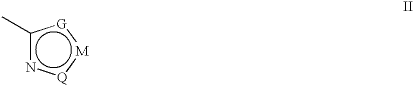
wherein G is O, S, N—R^{g};
M is N or C—R^{h}; or
Q is N or C—Rⁱ.

5. The method of claim 3, wherein Het-Y is 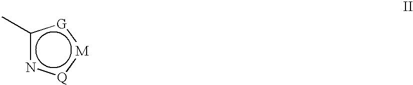
wherein G is N or C—R^{h};
M is N or C—R; and
Q is O, S, or N—R^{g}.

6. The method of claim 3, wherein Het-Y is

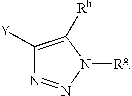

7. The method of claim 1, wherein Het-Y is 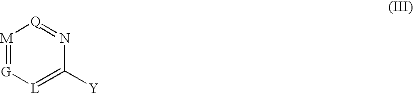
wherein Q, M, G and L are independently N⁻, C—R^{j}, C—R^{k}, C—R^{l}, or C—R^{m}, with the proviso that there are 1 to 3 N atoms in the ring, wherein R^{j}, R^{k}, R^{l} and R^{m} are
a. independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl (which alkyl can be substituted with alkyloxyimino), cycloalkyl, dialkylamino, halo, hydroxy, (C₂-C₆)hydroxyalkyl, mercapto, nitro, sulfamoyl, sulfonic acid, alkylthio, alkylsulfonyl, alkylsulfinyl, alkylsulfonamido, trifluoromethyl, morpholin-4-yl, 4-[C₆ or C₁₀]arylpiperidin-1-yl, 4-[C₆ or C₁₀]arylpiperazin-1-yl, thiomorpholin-4-yl, piperidin-1-yl, Ar*, Ar*-alkyl, Ar*-O, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or
b. where two of R^{j}, R^{k}, R^{l} or R^{m} are adjacent, together with their ring carbons form a C₅-C₇ fused cycloalkyl ring having up to two double bonds including the fused double bond of the Het group, which cycloalkyl ring can be substituted by one or more of the group consisting of alkyl-, alkoxycarbonyl-, amino-, aminocarbonyl-, carboxy-, fluoro-, or oxo- substituents, wherein multiple substituents are located on different carbon atoms of the cycloalkyl ring, except in the case of alkyl, alkoxycarbonyl, and fluoro substituents, which may be located on the same or different carbon atoms; or
c. where two of R^{j}, R^{k}, R^{l} and R^{m} are adjacent, together with their ring carbons form a fused C₆ or C₁₀ aryl; or
d. where two of R^{j}, R^{k}, R^{l} and R^{m} are adjacent, together with their ring carbons form a fused five to eight membered fused heterocycle, wherein the ring fusion is at a carbon-carbon bond of Het, wherein the fused heterocycle consists of ring atoms selected from the group consisting of carbon, nitrogen, oxygen, sulfur, or S(O)ₙ, wherein n is 1 or 2; or
e. where two of R^{j}, R^{k}, R^{l} and R^{m} are adjacent, together with their ring carbons form a fused 5- or 6-membered heteroaryl ring containing at least one and up to three atoms of N for the 6-membered fused heteroaryl rings and from one to three atoms of N or one atom of O or S and zero to two atoms of N for the 5-membered fused heteroaryl rings.

8. The method of claim 7,
a. wherein L is N;
b. G is C—R^{j};
C. M is C—R^{k};
d. Q is C—R^{l}; and
e. Y is amino, amino(C₁-C₅)alkyl, or aminophenyl, wherein the amino of all three groups can be substituted with
(1) Ar*,
(2) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂ or
(3) formyl or alkanoyl.

9. The method of claim 7,
a. wherein L and Q are N;
b. G is C—R^{j};
c. M is C—R^{k}; and
d. Y is amino, amino(C₁-C₅)alkyl, or aminophenyl, wherein the amino of all three groups can be substituted with
(1) Ar*,
(2) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂ or
(3) formyl or alkanoyl.

10. The method of claim 3,
a. wherein Q is N;
b. G is N—R^{g};
c. M is C—R^{h}; and
d. Y is amino, amino(C₁-C₅)alkyl, or aminophenyl, wherein the amino of all three groups can be substituted with
(1) Ar*,
(2) Ar*-Z—, Ar*-alkyl-Z—, Ar*-Z-alkyl-, Ar*-amino-Z—, Ar*-aminoalkyl-Z— or Ar*-oxyalkyl-Z—, wherein Z is a carbonyl or S(O)₂ or
(3) formyl or alkanoyl.
